SEQUENCE LISTING (14 sequences)

SEQ ID NO: 1 - DNA, 41 bp, Artificial Sequence
  apatamer
atggttgtgtttttttttgtgtggcttcgtatgttgttgcg

SEQ ID NO: 2 - DNA, 28 bp, Artificial Sequence
  apatamer
ccaaaaaaacaccaaaaacaaaaccact

SEQ ID NO: 3 - DNA, 28 bp, Artificial Sequence
  apatamer
agtggttttgtttttggtgtttttttgg

SEQ ID NO: 4 - DNA, 41 bp, Artificial Sequence
  apatamer
gcggtctgactgggtagcagtagaaggcgtcggtcccgggt

SEQ ID NO: 5 - DNA, 41 bp, Artificial Sequence
  apatamer
acccgggaccgacgccttctactgctacccagtcagaccgc

SEQ ID NO: 6 - DNA, 41 bp, Artificial Sequence
  apatamer
cacagccgcacacacagaactccaggatcttaccgcctcat

SEQ ID NO: 7 - DNA, 41 bp, Artificial Sequence
  apatamer
atgaggcggtaagatcctggagttctgtgtgtgcggctgtg

SEQ ID NO: 8 - DNA, 73 bp, Artificial Sequence
  apatamer
tacagcaccacagaccatggttgtgtttttttttgtgtggcttcgtatgttgttgcgtgtttgtcttcctgcc

SEQ ID NO: 9 - DNA, 60 bp, Artificial Sequence
  apatamer
ggcaggaagacaaacaccaaaaaaacaccaaaaacaaaaccactggtctgtggtgctgta

SEQ ID NO: 10 - DNA, 60 bp, Artificial Sequence
  apatamer
tacagcaccacagaccagtggttttgtttttggtgtttttttggtgtttgtcttcctgcc

SEQ ID NO: 11 - DNA, 73 bp, Artificial Sequence
  apatamer
ggcaggaagacaaacagcggtctgactgggtagcagtagaaggcgtcggtcccgggtggtctgtggtgctgta

SEQ ID NO: 12 - DNA, 73 bp, Artificial Sequence
  apatamer
tacagcaccacagaccacccgggaccgacgccttctactgctacccagtcagaccgctgtttgtcttcctgcc

SEQ ID NO: 13 - DNA, 73 bp, Artificial Sequence
  apatamer
ggcaggaagacaaacacacagccgcacacacagaactccaggatcttaccgcctcatggtctgtggtgctgta

SEQ ID NO: 14 - DNA, 73 bp, Artificial Sequence
  apatamer
tacagcaccacagaccatgaggcggtaagatcctggagttctgtgtgtgcggctgtgtgtttgtcttcctgcc

(19) United States
(12) Patent Application Publication
(10) Pub. No.: US 2016/0097101 A1
(43) Pub. Date: Apr. 7, 2016

(54) APTAMER SPECIFIC TO COLORECTAL CANCER CELL AND APPLICATION THEREOF

(71) Applicant: National Tsing Hua University, Hsinchu (TW)

(72) Inventors: Gwo-Bin LEE, Hsinchu (TW); Lien-Yu HUNG, Hsinchu (TW); Chih-Hung WANG, Hsinchu (TW)

(21) Appl. No.: 14/595,766

(22) Filed: Jan. 13, 2015

(30) Foreign Application Priority Data
Oct. 7, 2014 (TW) 103134849

(51) Int. Cl. C12Q 1/68

(52) U.S. Cl. CPC C12Q 1/6886; C12Q 2600/118; C12Q 2600/158

(57) ABSTRACT

The present invention provides an aptamer specific to colorectal cancer cells and comprises a nucleotide sequence selected from the group consisting of Seq ID: NO. 1 to Seq ID: NO. 14. The aptamer of the present invention specific to colorectal cancer cell may be applied in colorectal cancer detection and provide a detection method having non-invasive, fast and convenient properties.

### BACKGROUND OF THE INVENTION

1. Field of the Invention

The present invention relates to a novel aptamer nucleotide sequence and uses thereof, particularly to an aptamer nucleotide sequence specific to colorectal cancer cell and uses thereof.

2. Description of the Prior Art

Cancer is one of the leading causes of death in humans. Colorectal cancer (CRC) is the most frequently diagnosed cancer claiming about 700,000 lives every year. The earlier the cancer is diagnosed, a significantly increase in the five-year survival rate of the patients is observed. For example, patients diagnosed with stage I CRC have a five-year survival rate higher than 90%. The number drops to less than 10% at stage IV reflecting the importance of early diagnose of CRC.

Traditional methods for CRC diagnosis commonly involved invasive approaches such as digital rectal examination, proctoscopy, flexible sigmoidoscopy, and colofibroscopy. These endoscopy-based methods are generally accurate tests offering advantages such as direct observation of polyps and therefore are wildly used in hospitals. Like other invasive diagnosis methods, these approaches possess a higher risk and can result in discomfort.

Fecal occult blood test (FOBT) is a cheap and simple to perform method, although the false-positive result is generally high. Furthermore, serological tests using carcinoembryonic antigen (CEA) and carbohydrate antigen 19-9 (CA19-9) as biomarkers for CRC diagnosis have also been performed. However, these markers are not specific enough for CRC early detection since patients with pancreatic cancer and lung cancer also show an increase of CEA and CA 19-9 values.

The advancement of cancer therapeutic technology has greatly improved the survival rates of patients with CRC, although recurrence of the cancer is still common. It is recognized now that a small fraction of cancer cells, named cancer stem cells (CSC), show distinct biological features from other cells in the cancer population. Cancer stem cells possess the ability of self-renewal, the capability of developing multiple cell lineages, and the potential of extensive proliferation. Cancer stem cells also display high drug resistance and are therefore difficult to eradicate. If therapies can be targeted against CSCs such that the tumor may lose its ability of growing and maintaining, then it may eventually lead to a complete cure. Cancer stem cells have been identified in CRC, and the cells are known to contribute to metastasis in the patients after receiving chemotherapy. In order to detect or isolate colorectal cancer stem cells (CR-CSCs), certain cell surface molecules including CD44, CD133 (Prominin-1), and EpCAM have been used as biomarkers of CR-CSCs.

However, these molecules are also present in other types of CSCs and do not have sufficient specificity for CR-CSC detection. Therefore, the development of a technology to efficiently identify novel specific biomarkers for CR-CSC and CRC cells detection will contribute greatly in diagnosis and treatment of CRC.

### SUMMARY OF THE INVENTION

The present invention is directed to providing a novel aptamer nucleotide sequence, which is specific to colorectal cancer cell and may be applied in colorectal cancer detection and provide a detection method having non-invasive, fast and convenient properties.

According to an embodiment, an aptamer specific to colorectal cancer cells comprises a nucleotide sequence selected from the group consisting of Seq ID: NO. 1 to Seq ID: NO. 14.

According to another embodiment, a method for colorectal cancer detection comprises contacting an aptamer specific to colorectal cancer cells to a test sample for binding reaction, wherein the aptamer comprises a nucleotide sequence selected from the group consisting of Seq ID: NO. 1 to Seq ID: NO. 14; and calculating a binding ratio between the aptamer and the test sample.

Other advantages of the present invention will become apparent from the following descriptions taken in conjunction with the accompanying drawings wherein certain embodiments of the present invention are set forth by way of illustration and examples.

### BRIEF DESCRIPTION OF THE DRAWINGS

The foregoing aspects and many of the accompanying advantages of this invention will become more readily appreciated as the same becomes better understood by reference to the following detailed descriptions, when taken in conjunction with the accompanying drawings, wherein:

FIG. 1 is a flowchart diagram illustrating for screening aptamers specific to colorectal cancer cells according to one embodiment of the present invention.

FIG. 2 is experimental data showing the cellular characteristics of the cell lines used in the present invention.

FIG. 3 is experimental data showing the specificity test of aptamer nucleotide sequences to colorectal cancer cell and colorectal cancer stem cell.

FIG. 4a and FIG. 4b are experimental data showing the capture ability of aptamer nucleotide sequences to colorectal cancer cell and colorectal cancer stem cell.

### DESCRIPTION OF THE PREFERRED EMBODIMENT

The present invention is further detailed with following preferred embodiments in accompany with drawings. It should be noted that the following experimental data of various disclosed embodiments is used for ease of explanation the technical features of the present invention and not intended to limit to the aspects which may be implemented.

Generally speaking, an aptamer refers to an oligonucleotide or a peptide chain bound to a specific target, such as small molecule compounds, proteins, nucleic acids, cells, tissues or organs. The aptamer is obtained from screening out of a large number of random sequences and may be applied in research and development of macromolecular drugs as well as study of clinical diagnosis.

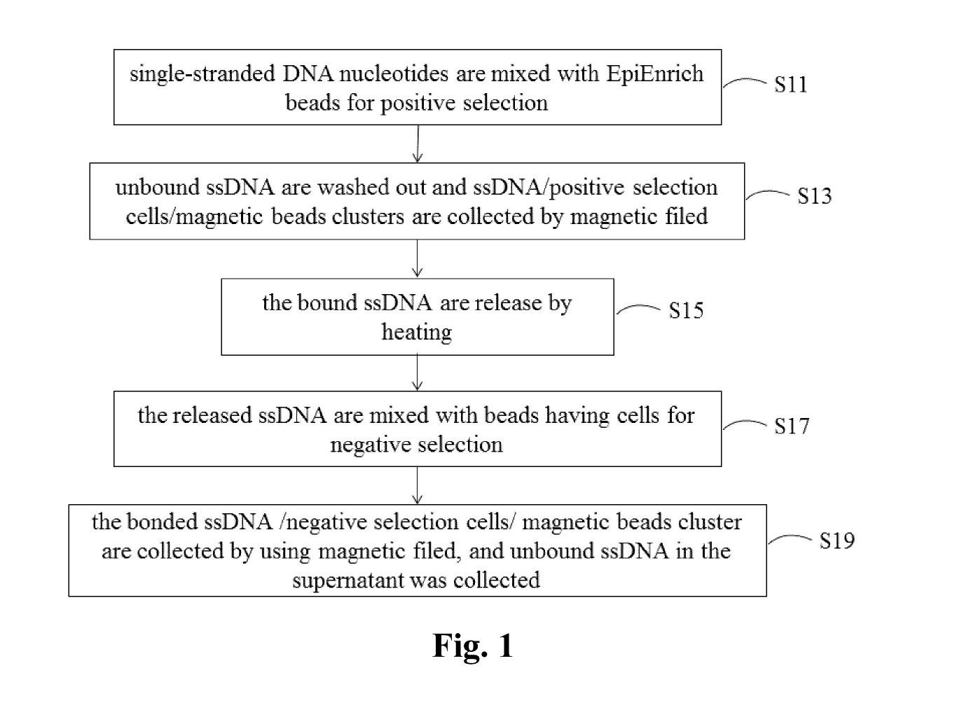

The present invention provides nucleotide aptamers that specifically bind to colorectal cancer cells and sequence and are obtained by following screening steps illustrated in FIG. 1. First, An approach for screening biomarkers for CRC, systematic evolution of ligands by exponential enrichment (SELEX), from a single-stranded DNA library is used in accompany with Epithelial Enrich immunomagnetic beads (EpiEnrich beads). In step S11, the single-stranded DNA nucleotides are mixed with EpiEnrich beads for positive selection. Here, in one embodiment, the cells used for positive selection may be colorectal cancer cell line HCT-8. Alternatively, in another embodiment, the cells used for positive selection may be colorectal cancer stem cell (CR-CSC) that are isolated by flow cytometry and enriched by suspension culture. ssDNA having binding affinity with cells, namely the colorectal cancer cell line HCT-8 or colorectal cancer stem cell CR-CSC, bind to these cells for positive selection. Then, in step S13, unbound ssDNA are washed out and ssDNA/positive selection cells/magnetic beads clusters are collected by magnetic field to complete the primary screening step.

Then, in step S15, the collected ssDNA/positive selection cells/magnetic beads clusters are heated to release the bound ssDNA. In step S17, the released ssDNA are co-cultured with beads having cells for negative selection, which are different from those cell listed above. Finally, the bound ssDNA/negative selection cells/magnetic beads cluster are collected by using magnetic field, and unbound ssDNA in the supernatant was collected, as illustrated in step S19. In this way, ssDNA having binding specificity and affinity to positive selection cells, namely HCT-8 or CR-CSC, are collected to complete the second step of the screening.

Then, ssDNA obtained from the above method are amplified by polymerase chain reaction (PCR) and screened from a plurality of cycles, preferably, 5 cycles.

After cycles of screening, the screened ssDNA are harvested for cloning and sequencing.

According to the above-described embodiments of the present invention, nucleotide sequences having binding specificity and affinity to HCT-8 or CR-CSC are screened, as listed in Seq ID: NO. 1 to Seq ID: NO. 7.

In another embodiment of the present invention, for further experiment, some primer sequences may be added to the 5′ end and 3′ end of the above-described screened nucleotide sequences having binding specificity and affinity to HCT-8 or CR-CSC. The nucleotide sequences with added primer are shown as Seq ID: NO. 8 to Seq ID: NO. 14. Exemplary nucleotide sequences of Seq ID: NO. 8 to Seq ID: NO. 14 are test in the later; however, the present invention is not thus limited but includes set forth nucleotide sequences such as Seq ID: NO. 1 to Seq ID: NO. 7.

### Cellular Qualitative Image Analysis

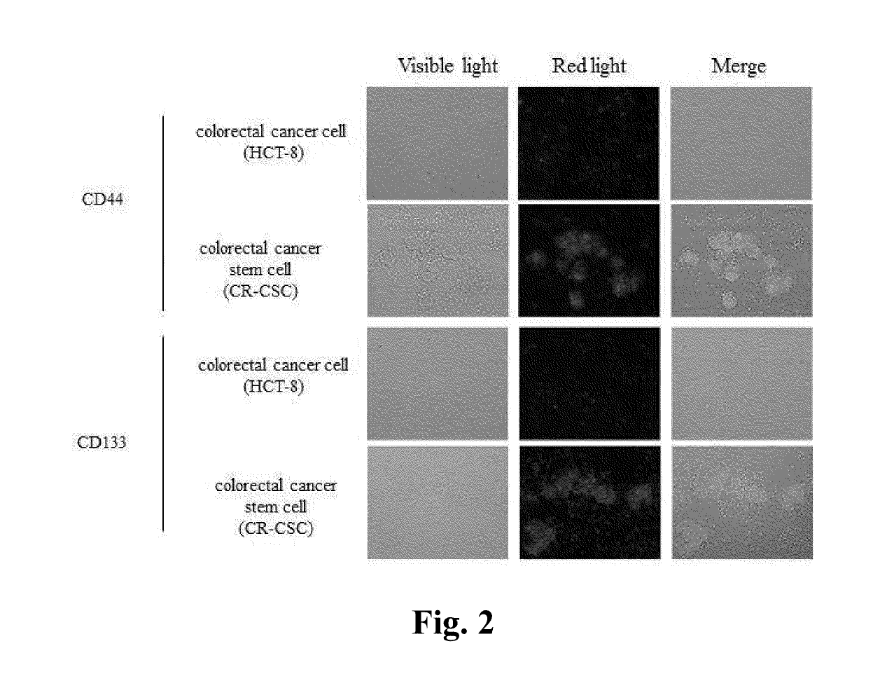

In one embodiment, cellular qualitative image analysis is performed to identify the binding specificity of nucleotide sequences of Seq ID: NO. 8 to Seq ID: NO. 14 to colorectal cancer cells. First, stem cell properties of HCT-8 or CR-CSC are identified via cancer stem cell surface molecules CD44 and CD133. Wherein, HCT-8 and CR-CSC were tested with anti-CD44 and CD133 antibodies having red fluorescence, respectively, as illustrated in FIG. 2. The binding of anti-CD44 and CD 133 antibodies having red fluorescence is only observed in CR-CSC, indicating that the CR-CSC used in the present invention does have properties of cancer stem cells.

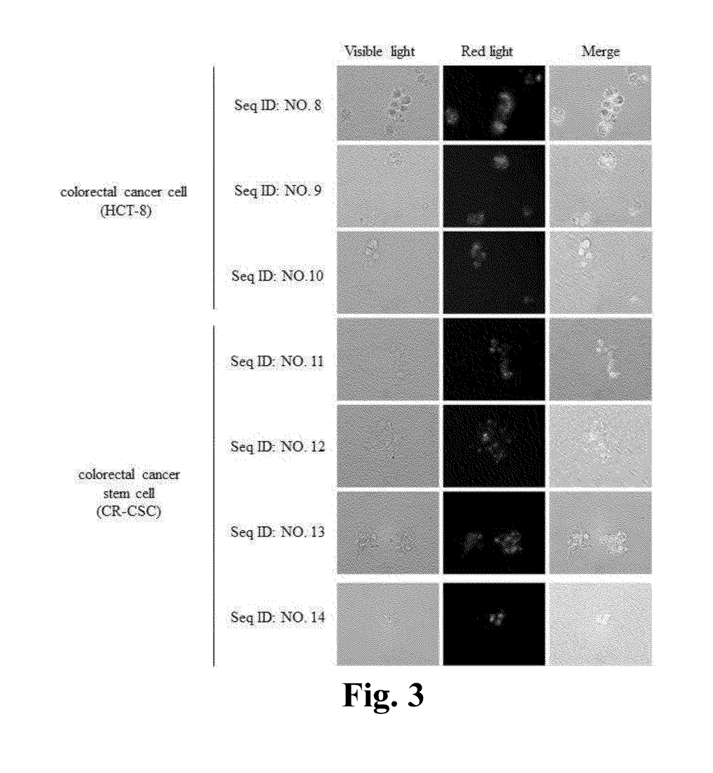

Nucleotide sequences of Seq ID: NO. 8 to Seq ID: NO. 14 are labeled with green fluorescence (FAM fluorescence) in 5 'end and respectively mixed to HCT-8 and CR-CSC so as to confirm the binding specificity of the nucleotide sequences to the above cell lines, as illustrated in FIG. 3. Wherein, colorectal cancer cell line HCT-8 is found to be bound with Seq ID: NO. 8 to Seq ID: NO. 10 having green fluorescence, indicating that Seq ID: NO. 8 to Seq ID: NO. 10 are provided with specific binding ability to HCT-8. Colorectal cancer stem cell CR-CSC is found to be bound with Seq ID: NO. 11 to Seq ID: NO. 14 having green fluorescence, indicating that Seq ID: NO. 11 to Seq ID: NO. 14 are provided with specific binding ability to CR-CSC.

### Capturing Ability Analysis

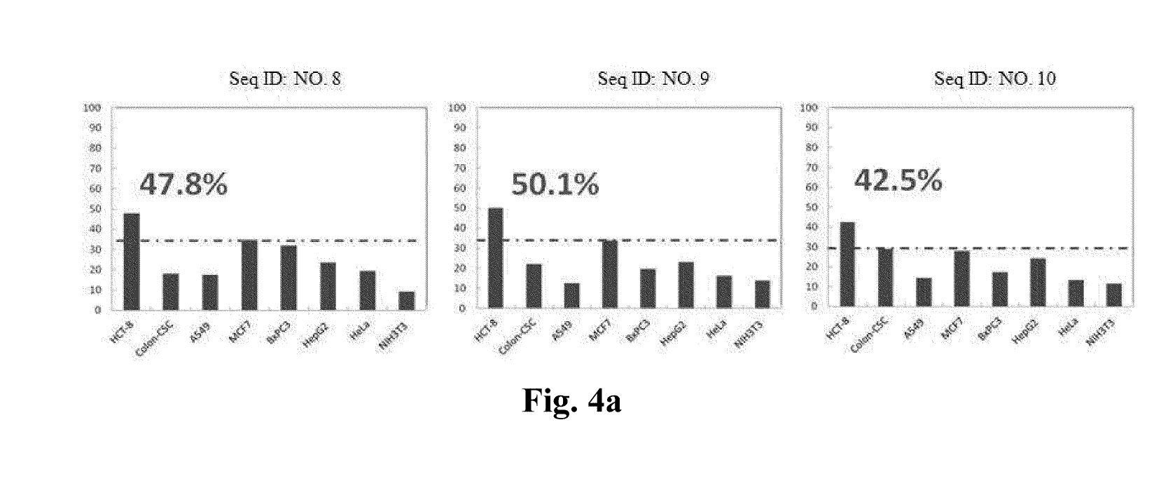

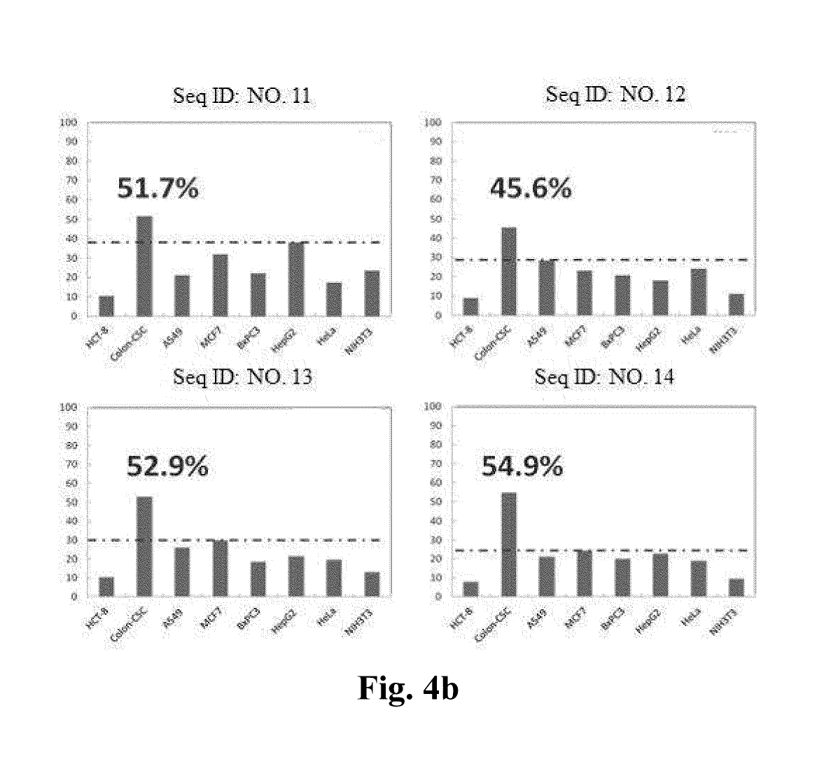

In one embodiment, the nucleotide sequences of Seq ID: NO. 8 to Seq ID: NO. 14 are respectively bonded to the beads and mixed with different cell lines, respectively, including colon colorectal cancer cells (HCT-8), colorectal cancer stem cells (CR-CSC), human lung adenocarcinoma cells (A549), human breast cancer cells (MCF7), human in situ pancreatic cancer cells (BxPC-3), human hepatoma cells (HepG2), human cervical cancer cells (Hela), and mouse fibroblasts (NIH-3T3). The beads having nucleotide sequences are mixed with each cell line at room temperature for 25 rpm 30 minutes, and the cell number of each cell line is 2×10⁵. After washing for several times, the number of cells captured with magnetic beads having the above nucleotide sequences is calculated, and the results are illustrated in FIG. 4a and FIG. 4b.

The capture rate of HCT-8 by Seq ID: NO. 8 to Seq ID: NO. 10 are 47.8%, 50.1%, and 42.5%, respectively. The capture rate of CR-CSC by Seq ID: NO. 11 to Seq ID: NO. 14 are 51.7%, 45.6%, 52.9% and 54.9%, respectively. Therefore, the nucleotide sequences of the present invention provide higher recognition rate to colorectal cancer cell and colorectal cancer stem cell, than that to other tumor cells.

In summary, the aptamers of the present invention having the nucleotide sequences Seq ID: NO. 8 to Seq ID:. NO. 14 are provided with higher specificity and affinity to colorectal cancer cell and colorectal cancer stem cell, respectively and may contribute to early detection of colorectal cancer and more convenient and faster methods for detecting cancer.

While the invention can be subject to various modifications and alternative forms, a specific example thereof has been shown in the drawings and is herein described in detail. It should be understood, however, that the invention is not to be limited to the particular form disclosed, but on the contrary, the invention is to cover all modifications, equivalents, and alternatives falling within the spirit and scope of the appended claims.

What is claimed is:

1. An aptamer specific to colorectal cancer cells, comprising a nucleotide sequence selected from the group consisting of Seq ID: NO. 1 to Seq ID: NO. 14.

2. The aptamer specific to colorectal cancer cells according to claim 1, wherein the colorectal cancer cells comprise colorectal cancer stem cells.

3. The aptamer specific to colorectal cancer cells according to claim 1, consisting of the nucleotide sequence selected from the group consisting of Seq ID: NO. 1 to Seq ID: NO. 14.

4. The aptamer specific to colorectal cancer cells according to claim 1, consisting of the nucleotide sequence selected from the group consisting of Seq ID: NO. 8 to Seq ID: NO. 14.

5. A method for colorectal cancer detection, comprising:
contacting an aptamer specific to colorectal cancer cells to a test sample for binding reaction, wherein the aptamer comprises a nucleotide sequence selected from the group consisting of Seq ID: NO. 1 to Seq ID: NO. 14; and
calculating a binding ratio between the aptamer and the test sample.

6. The method for colorectal cancer detection according to claim 5, wherein the colorectal cancer cells comprise colorectal cancer stem cells.

7. The method for colorectal cancer detection according to claim 5, wherein the aptamer consists of the nucleotide sequence selected from the group consisting of Seq ID: NO. 1 to Seq ID: NO. 14.

8. The method for colorectal cancer detection according to claim 5, wherein the aptamer consists of the nucleotide sequence selected from the group consisting of Seq ID: NO. 8 to Seq ID: NO. 14.

9. The method for colorectal cancer detection according to claim 5, wherein the test sample comprises cells or tissues.

10. The method for colorectal cancer detection according to claim 5, further comprising:
determining if the test sample comprises colorectal cancer cells based on the binding ratio.
